(19) United States
(12) Patent Application Publication
(10) Pub. No.: US 2026/0185997 A1
(43) Pub. Date: Jul. 2, 2026

(54) IMMOBILIZED AND STABILIZED LOO_GFP FLUORESCENT BIOSENSORS WITH AN ENHANCED DYNAMIC RANGE

(71) Applicant: The Texas A&M University System, College Station, TX (US)

(72) Inventors: Sarah Bondos, College Station, TX (US); Rebecca Booth, College Station, TX (US); Amanda Jons, College Station, TX (US)

(73) Assignee: The Texas A&M University System, College Station, TX (US)

(21) Appl. No.: 19/131,562

(22) PCT Filed: Nov. 21, 2023
(86) PCT No.: PCT/US2023/080726

§ 371 Date: May 21, 2025

Related U.S. Application Data
(60) Provisional application No. 63/384,476, filed on Nov. 21, 2022.

(51) Int. Cl. G01N 33/68; C07K 14/435

(52) U.S. Cl. CPC G01N 33/6845; C07K 14/43581; C07K 14/43595; C07K 2319/00; G01N 2333/43573; G01N 2333/43595

(57) ABSTRACT

Disclosed are leave-one-out GFP (LOO_GFP) biosensors immobilized in Ubx (Drosphila protein Ultrabithorax) that form immobilized and stabilized fluorescent protein biosensor systems (LOO_GFP-Ubx systems). These LOO_GFP-Ubx systems have an enhanced fluorescent signaling to noise ratio. Also disclosed are methods of making LOO_GFP-Ubx systems and methods of using such systems to detect analyte. In preferred embodiments, the LOO_GFP-Ubx system has the eighth β strand or eleventh f strand of OPT GFP left out.

### RELATED APPLICATION

The present disclosure claims priority to U.S. Provisional Patent Application Ser. No. 63/384,476, filed Nov. 21, 2022, which is incorporated by reference herein in its entirety.

### GOVERNMENT SUPPORT

This invention was made with government support under Grant No. R01GM099827 awarded by the National Institutes of Health. The government has certain rights in the invention.

### BACKGROUND OF THE INVENTION

### 1. Field of the Invention

The invention generally relates to the field of biosensors. More specifically, the invention relates to immobilized and stabilized leave-one-out green fluorescent protein biosensor systems with enhanced dynamic range.

### 2. Description of Related Art

Biosensors are devices that rely on living organisms and/or biological molecules to detect specific substances or conditions, often disease agents or environmental contaminants. Biosensors must specifically recognize an analyte and transduce a signal upon analyte binding that ideally can be detected using simple, portable equipment. Furthermore, biomaterials must be biocompatible if used in vivo. Proteins offer a unique opportunity to achieve these goals: they can bind other molecules with unparalleled specificity, they can create visible, electronic, or chemical signals, and they are a natural component of all life.

Protein-fragment complementation assays (PCAs) provide a mechanism to link analyte binding with signal generation by a protein. In this approach, a protein is divided into two chains which individually lack function but reconstitute their ability to generate a signal when recombined. Many PCAs have been designed to investigate protein-protein interactions in a wide variety of fields (see, e.g., Romei, M. G., Boxer, S. G., “Split Green Fluorescent Proteins: Scope, limitations, and outlook,” Annual Review of Biophysics 2019, 48 (1), 19-44). As an example, split green fluorescent protein (GFP) systems offer the advantage of a self-reporting fluorescent readout that requires no reagents. These properties make on-site detection and analysis possible and facile, requiring only a blue light source and an orange high-pass filter which can be adapted for use with a standard smart phone.

However, one problem with the split complementation assay is that division of a protein may destabilize the fragments, leading to lower activity, aggregation, or proteolysis. In the case of GFP, the fluorescent yield can be reduced even in the reconstituted protein.

Current solutions include incorporating such proteins into materials by either (1) covalently crosslinking them to the surface of the materials after assembly or (2) physically encapsulating them within the matrix of the materials. However, both methods often result in the loss of functional proteins. With regards to the first method, the crosslinking agent can inactivate the appended protein or remain embedded in the materials, making them toxic to cells. With regards to the second method, physically encapsulated proteins may diffuse out of the materials or become inactive due to the harsh chemical environment frequently used to stimulate the assembly of the proteins into materials.

An attractive alternative for materials composed of recombinant proteins, such as GFP, is the use of protein fusions, in which a gene encoding a self-assembling protein is fused to a gene encoding a functional protein, resulting in a single polypeptide containing the sequences of both proteins. The benefits of this alternative method includes the ability to: (1) achieve stoichiometric levels of functionalization; (2) obtain materials assembly and functionalization in one step; (3) avoid the loss of functional protein due to diffusion; (4) pattern functional proteins within the materials; and (5) avoid toxic byproducts associated with chemical crosslinking.

However, it is critical that functional proteins maintain their stability and properties when incorporated into materials via gene fusion. Certain functional proteins may not be good candidates for incorporation via gene fusion because they are unstable, insoluble, or too large/multimeric. Thus, this technique has most often been used only for short peptides and a limited number of small, monomeric, single-domain proteins.

Accordingly, a need has arisen for stable split GFP-based biosensors that retain their mechanical and functional properties. The present invention addresses this need with leave-one-out GFP (LOO_GFP) biosensors immobilized in Ubx (Drosphila protein Ultrabithorax) materials via gene fusion, resulting in a biosensor having improved fluorescent signal and reduced background fluorescence (i.e., noise).

All of the subject matter discussed in the Background is not necessarily prior art and should not be assumed to be prior art merely as a result of its discussion in the Background section. Along these lines, any recognition of problems in the prior art discussed in the Background or associated with such subject matter should not be treated as prior art unless expressly stated to be prior art. Instead, the discussion of any subject matter in the Background should be treated as part of the inventor's approach to the particular problem, which in and of itself, may also be inventive.

### SUMMARY OF THE INVENTION

The following presents a simplified summary of the disclosure in order to provide a basic understanding of some aspects of the disclosure. This summary is not an exhaustive overview of the disclosure. It is not intended to identify key or critical elements of the disclosure or to delineate the scope of the disclosure. Its sole purpose is to present some concepts in a simplified form as a prelude to the more detailed description that is discussed later.

Immobilization of LOO_GFP within protein materials, for example Drosophila melanogaster Ultrabithorax (Ubx) (i.e., a “LOO_GFP-Ubx” system) sterically blocks non-specific LOO_GFP oligomerization and stabilizes the LOO_GFPs by effectively encasing the LOO_GFP cargo protein at high density within the materials matrix. As a result, LOO_GFP-Ubx systems provide improved biosensor function, increasing the fluorescent signal relative to LOO_GFP, preventing LOO_GFP oligomerization and thus reducing associated background fluorescence, and increasing the stability and thus lifetime of the biosensor, potentially enabling the biosensor to be deployed without maintaining a cold chain for storage. By both increasing the signal and decreasing the background noise, immobilization of LOO_GFP in Ubx materials increases the dynamic range of the LOO_GFP biosensor.

An embodiment of this disclosure includes a composition comprising: a protein-fragment complementation assay wherein at least one component is immobilized with ultrabiothorax (Ubx) materials, wherein the protein-fragment complementation assay comprises a fluorescent protein, such as a green fluorescent protein or variant thereof, for example OPT GPT.

An embodiment of this disclosure includes a composition comprising: a protein-fragment complementation assay wherein at least one component is immobilized with ultrabiothorax (Ubx) materials, wherein the protein-fragment complementation assay comprises a fluorescent protein, such as a green fluorescent protein or variant thereof, for example OPT GPT, wherein Ubx is fused to a component of the protein-fragment complementation assay.

An embodiment of this disclosure includes a composition comprising: a protein-fragment complementation assay wherein at least one component is immobilized with ultrabiothorax (Ubx) materials, wherein the protein-fragment complementation assay is a fluorescent protein, such as a split fluorescent protein system, wherein Ubx is fused to a component of the protein-fragment complementation assay. In addition, the fluorescent protein may be a green fluorescent protein or variant thereof, for example OPT GPT.

An embodiment of this disclosure includes a composition comprising: a protein-fragment complementation assay wherein at least one component is immobilized with ultrabiothorax (Ubx) materials, wherein the protein-fragment complementation assay is a fluorescent protein, such as a split fluorescent protein system, wherein Ubx is fused to a component of the protein-fragment complementation assay, such as to part of the split fluorescent protein system.

An embodiment of this disclosure includes a composition comprising: a protein-fragment complementation assay wherein at least one component is immobilized with ultrabiothorax (Ubx) materials, wherein the protein-fragment complementation assay comprises a fluorescent protein, wherein Ubx is fused to a component of the protein-fragment complementation assay, such as LOO_GFP. Preferably, Ubx is fused to LOO8_GFP or LOO11_GFP.

An embodiment of this disclosure includes a method of manufacturing LOO_GFP-Ubx fusion protein suitable for a biosensor comprising: co-expressing a LOO_GFP-Ubx fusion protein and a left-out peptide in a first host cell; extracting protein from the first host cell; incubating the extracted protein in a buffer reservoir to support film formation; pulling fibers from the film; air drying the fibers; and washing the fibers with a denaturing wash to remove the left-out peptide. Preferably, the LOO_GFP-Ubx fusion protein is LOO8_GFP-Ubx and the left-out peptide is s8, or the LOO_GFP-Ubx and the left-out peptide are LOO11_GFP-Ubx and s11, respectively. In addition, LOO_GFP-Ubx fusion protein and the left-out peptide may be separately expressed from plasmid vectors or co-expressed from the same plasmid vector. Furthermore, the denaturing wash preferably includes a detergent and a concentration of 1M NaCl to 1.25M NaCl.

An embodiment of this disclosure includes a method of manufacturing LOO_GFP-Ubx fusion protein suitable for a biosensor comprising: co-expressing a LOO_GFP-Ubx fusion protein and a left-out peptide in a first host cell; extracting protein from the first host cell; incubating the extracted protein in a buffer reservoir to support film formation; pulling fibers from the film; air drying the fibers; and washing the fibers with a denaturing wash to remove the left-out peptide. The method may further comprise: expressing Ubx in a second host cell; extracting Ubx protein from the second host cell, and incubating the extracted Ubx protein with the extracted LOO_GFP-Ubx fusion protein in the buffer reservoir in a ratio of 10 percent LOO_GFP-Ubx fusion protein to 90% Ubx protein. Preferably, the LOO_GFP-Ubx is LOO8_GFP-Ubx and the left-out peptide is s8, or the LOO_GFP-Ubx and the left-out peptide are LOO11_GFP-Ubx and s11, respectively. In addition, the denaturing wash preferably includes a detergent and a concentration of 1M NaCl to 1.25M NaCl.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary, to employ concepts of the various patents, applications, and publications as identified herein to provide yet further embodiments. Other features, objects, and advantages will be apparent from the description, the drawings, and the claims.

### BRIEF DESCRIPTION OF THE DRAWINGS

The disclosure may be understood by reference to the following description taken in conjunction with the accompanying drawings, in which:

FIG. 1 shows a ribbon diagram of parent protein OPT GFP, comprising 11 β-strands, rotated to visualize a chromophore (shown as a stick structure).

FIG. 2 shows a ribbon diagram of LOO_GFP without a chromophore, depicting a cavity created by the removal of one of the β-strands. Residues surrounding the cavity are indicated by arrows with stars, and the removed β-strand is shown to the right.

FIG. 3 schematically shows processing of a LOO_GFP system. Here, LOO_GFP is co-expressed with its left-out peptide, termed “priming” peptide (shaded dark gray) to ensure chromophore maturation, proper folding, and fluorescence (shown as a starburst). Denaturing conditions remove the left-out peptide, preventing fluorescence. Addition of synthetic peptide, termed “target” peptide, which may have the same amino acid sequence of the left-out peptide, allows rebinding and reconstitution of fluorescence.

FIG. 4 schematically shows oligomerization of LOO_GFP monomers, which causes high background fluorescence and reduced signal-to-noise ratio. Ligand binding disrupts the oligomers.

FIG. 5 schematically shows a LOO8_GFP-Ubx fusion protein. LOO8_GFP-Ubx fusion protein may comprise a N-terminal His tag, a LOO8_GFP protein, Ubx, and GS linkers. An untagged s8 peptide (NGIKANFTVRHNV) is shown below and corresponds to OPT GFP residues 159-171.

FIG. 6 schematically shows conditions for removing fluorescence in consideration of the charge distribution of the proteins and size of the materials. For 10% LOO8_GFP-Ubx materials of 10-20 μm diameter, a pH 2.0 denaturing buffer with 1.25 M NaCl and 0.1% SDS was used. A first wash with PBS raises the pH back to 8.0 and does not add any additional salt or detergent. A second and third wash shield charge interactions with 1.25 M NaCl and use CHAPS to remove the SDS. Finally, synthetic s8 is added to recover fluorescence.

FIG. 7 shows a comparison of plain Ubx fibers (left) and s8: LOO8_GFP-Ubx fibers (right) acquired using confocal microscopy to track fluorescence emanating from dityrosine bonds (left of each group), differential contrast microscopy (middle of each group), and light microscopy (right of each group). Like plain Ubx fibers, the s8: LOO8_GFP-Ubx fibers lack pits, thin segments, or fraying, features which would have indicated poor assembly. Scale bar is 20 μm for all images. Brightness and contrast of images were adjusted equally to enhance visual quality.

FIG. 8 shows similar excitation and emission spectra for LOO8_GFP-Ubx and EGFP-Ubx. All samples had a maximum excitation peak of 488 nm, whereas the emission peak for all monomer samples (monomers of EGFP, EGFP-Ubx, and LOO8-Ubx as well as EGFP-Ubx and LOO8_GFP-Ubx fibers) had a maximum of 510 nm. The emission peak for all fiber samples had a maximum of 515 nm. Spectra of monomers are reported using dotted or dashed lines, whereas fiber spectra are represented by solid lines. Peak maxima were normalized to 1 to compare the shape and position of spectra.

FIGS. 9A-9B show the effect of various conditions for removing priming peptide s8 on background fluorescence. Fluorescent intensity data was collected from confocal microscopy images of treated and untreated fibers. Data was normalized against the blue fluorescence and diameter of each fiber and is reported as “Relative Fluorescent Intensity”. Three measurements from each fiber image were taken to get an average intensity value for each sample. The dashed line indicates the ambient signal in the absence of sample.

FIG. 9A shows the fluorescent intensity of 100% LOO8_GFP-Ubx fibers relative to untreated fiber intensity (control) after a 5 second pH 2 glycine buffer wash with or without synthetic s8 added, or a 15-minute 4.5 M GdnHCl wash with or without synthetic s8 added. All samples were washed in PBS following exposure to denaturant to allow LOO8_GFP-Ubx refolding and rebinding to any remaining peptide.

FIG. 9B shows relative fluorescent intensity of washed fibers after being denatured for varying amounts of time. All fluorescence is lost after 30 seconds and there is no significant difference in background measured between 30 seconds and 30 minutes.

FIG. 9C shows a scatter plot of fiber diameter versus green fluorescence relative to blue fluorescence emanating from dityrosine bonds, a measure of the quantity of Ubx materials. Although the relative fluorescent intensity is comparable to baseline for all samples, thicker fibers (>40 μm) have higher recovery of fluorescence in the absence of s8 after 10 minutes of denaturation than thinner fibers (<10 μm), indicating priming s8 is less able to escape the fiber structure.

FIG. 9D shows relative fluorescent intensity of 10% LOO8_GFP-Ubx fibers 10-20 μm in diameter before and after denaturing in pH buffer for 10 minutes and washing three times in pH 8 buffer for 10 minutes.

FIGS. 10A-10B show concentration dependence of fiber fluorescence.

FIG. 10A shows images of fibers comprising 100% LOO8_GFP-Ubx before and after priming s8 removal at pH 2 for 10 minutes. To the right of panel A, an illustration depicts 100% LOO8_GFP-Ubx incorporation.

FIG. 10B shows images of fibers comprising 10% LOO8_GFP-Ubx and 90% Ubx, which may prevent oligomerization and reduce background signal. These fibers still fluoresce green, albeit with less intensity before s8 removal. In this formulation, no fluorescence can be detected in the absence of s8. Arrows mark the position of the fiber. To the right of panel B, an illustration depicts 10% LOO8-Ubx incorporation.

FIGS. 11A-11B show enhanced stabilization of LOO8_GFP by fusion to Ubx.

FIG. 11A shows the fluorescent intensity of LOO8_GFP and LOO8_GFP-Ubx monomers normalized by concentration. LOO8_GFP-Ubx is 1.4 times as fluorescent as LOO8_GFP, a significant improvement (p<0.05).

FIG. 11B shows representative confocal microscopy images of fibers before (two images on left) and after (two images on right) being autoclaved for 20 minutes. The first image on the right shows some pitting in the fiber caused by autoclaving, but no significant loss of fluorescence.

FIGS. 12A-12B show the effect of various conditions for rebinding s8 peptide on fluorescence. Fluorescent intensity data was collected from confocal microscopy images of treated and untreated fibers and normalized against the blue fluorescence and diameter of each fiber to account for sample volume and Ubx packing density. Three measurements from each fiber image were taken to get an average intensity value for each sample. The dashed line indicates the ambient signal in the absence of sample.

FIG. 12A shows the fluorescent intensity of fibers relative to untreated fiber intensity (control) with and without synthetic s8 added after denaturing with pH 2 buffer containing 0.1% SDS and washing with buffer containing 0.1% CHAPS, pH 8. None of the samples showed recovery of fluorescence above the background of the microscope (gray dashed line).

FIG. 12B shows a depiction of LOO8_GFP (left) showing the negative surface charge (dark gray) around the binding pocket for s8 and areas of positive surface charge (lighter gray) on helix 3; areas of positive charge are further distinguished by arrows. Further shown is a depiction of the Ubx homeodomain showing the positive surface charge (dark gray) on the disordered N-terminal arm. Images are not to scale.

FIG. 12C shows the fluorescent intensity of fibers relative to untreated controls with (+) and without (−) synthetic s8 added after denaturing and washing with different concentrations of NaCl. Samples denatured and washed in buffers containing 1 M NaCl and 1.25 M NaCl showed significant increase in fluorescent intensity above the background.

FIG. 12D shows the fluorescent intensity of fibers relative to untreated controls with and without synthetic s8 added to fibers denatured with 1.25 M NaCl and 0.1% SDS and washed with 1.25 M NaCl and 0.1% CHAPS. Significance was determined by 2 sample t-test for equal means, *p<0.05, **p<0.001, ***p<0.000001.

BRIEF DESCRIPTION OF THE SEQUENCES

SEQ ID NO. 1 is the amino acid sequence of LOO8_GFP:
HEDGSVQLADHYQQNTPIGDGPVLLPDNHYLSTQTVLSKDPNEKRDHMVLLEFVTA
AGITHGMDELYKGGTGGSMASKGEELFTGVVPILVELDGDVNGHKFSVRGEGEGDA
TIGKLTLKFICTTGKLPVPWPTLVTTLTYGVQCFSRYPDHMKRHDFFKSAMPEGYVQ
ERTISFKDDGKYKTRAVVKFEGDTLVNRIELKGTDFKEDGNILGHKLEYNFNSHNVY
ITADKQK
SEQ ID NO. 2 is the amino acid sequence of LOO8_GFP-Ubx:
MGHHHHHHHHHHSSGHEDGSVQLADHYQQNTPIGDGPVLLPDNHYLSTQTVLSKD
PNEKRDHMVLLEFVTAAGITHGMDELYKGGTGGSMASKGEELFTGVVPILVELDGD
VNGHKFSVRGEGEGDATIGKLTLKFICTTGKLPVPWPTLVTTLTYGVQCFSRYPDHM
KRHDFFKSAMPEGYVQERTISFKDDGKYKTRAVVKFEGDTLVNRIELKGTDFKEDG
NILGHKLEYNFNSHNVYITADKQKGSNSYFEQASGFYGHPHQATGMAMGSGGHHD
QTASAAAAAYRGFPLSLGMSPYANHHLQRTTQDSPYDASITAACNKIYGDGAGAYK
QDCLNIKADAVNGYKDIWNTGGSNGGGGGGGGGGGGGAGGTGGAGNANGGNAA
NANGQNNPAGGMPVRPSACTPDSRVGGYLDTSGGSPVSHRGGSAGGNVSVSGGNG
NAGGVQSGVGVAGAGTAWNANCTISGAAAQTAAASSLHQASNHTFYPWMAIAGKI
RSDLTQYGGISTDMGKRYSESLAGSLLPDWLGTNGLRRRGRQTYTRYQTLELEKEF
HTNHYLTRRRRIEMAHALCLTERQIKIWFQNRRMKLKKEIQAIKELNEQEKQAQAQK
AAAAAAAAAAVQGGHLDQ
SEQ ID NO. 3 is the amino acid sequence of s8, i.e., the omitted
eigth β-strand of LOO8-GFP:
NGIKANFTVRHNV
SEQ ID NO. 4 is the amino acid sequence of LOO11_GFP:
GITHGMDELYKGGTGGSMASKGEELFTGVVPILVELDGDVNGHKFSVRGEGEGDAT
IGKLTLKFICTTGKLPVPWPTLVTTLTYGVQCFSRYPDHMKRHDFFKSAMPEGYVQE
RTIS
FKDDGKYKTRAVVKFEGDTLVNRIELKGTDFKEDGNILGHKLEYNFNSHNVYITAD
KQKNGIKANFTVRHNVSSGHEDGSVQLADHYQQNTPIGDGPVLLPDNHYLSTQTVL
SKDPNEKR
SEQ ID NO. 5 is the amino acid sequence of LOO11_GFP-Ubx:
MGHHHHHHHHHHSSGGITHGMDELYKGGTGGSMASKGEELFTGVVPILVELDGDV
NGHKFSVRGEGEGDATIGKLTLKFICTTGKLPVPWPTLVTTLTYGVQCFSRYPDHMK
RHDFFKSAMPEGYVQERTISFKDDGKYKTRAVVKFEGDTLVNRIELKGTDFKEDGNI
LGHKLEYNFNSHNVYITADKQKNGIKANFTVRHNVSSGHEDGSVQLADHYQQNTPI
GDGPVLLPDNHYLSTQTVLSKDPNEKRNSYFEQASGFYGHPHQATGMAMGSGGHH
DQTASAAAAAYRGFPLSLGMSPYANHHLQRTTQDSPYDASITAACNKIYGDGAGAY
KQDCLNIKADAVNGYKDIWNTGGSNGGGGGGGGGGGGGAGGTGGAGNANGGNA
ANANGQNNPAGGM
PVRPSACTPDSRVGGYLDTSGGSPVSHRGGSAGGNVSVSGGNGNAGGVQSGVGVA
GAGTAWNANCTISGAAAQTAAASSLHQASNHTFYPWMAIAGKIRSDLTQYGGISTD
MGKRYSESLAGSLLPDWLGTNGLRRRGRQTYTRYQTLELEKEFHTNHYLTRRRRIE
MAHALCLTERQIKIWFQNRRMKLKKEIQAIKELNEQEKQAQAQKAAAAAAAAAAV
QGGHLDQ
SEQ ID NO. 6 is the amino acid sequence of s11, i.e., the omitted
eleventh β-strand of LOO11-GFP:
DHMVLLEFVTAA

### DETAILED DESCRIPTION OF THE INVENTION

Various illustrative embodiments of the disclosure are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

While the subject matter disclosed herein is susceptible to various modifications and alternative forms, specific embodiments thereof have been shown by way of example in the drawings and are herein described in detail. It should be understood, however, that the description herein of specific embodiments is not intended to limit the disclosure to the particular forms disclosed, but on the contrary, the intention is to cover all modifications, equivalents, and alternatives falling within the spirit and scope of the disclosure as defined by the appended claims.

### Leave-One-Out Split GFP (LOO_GFP) Systems

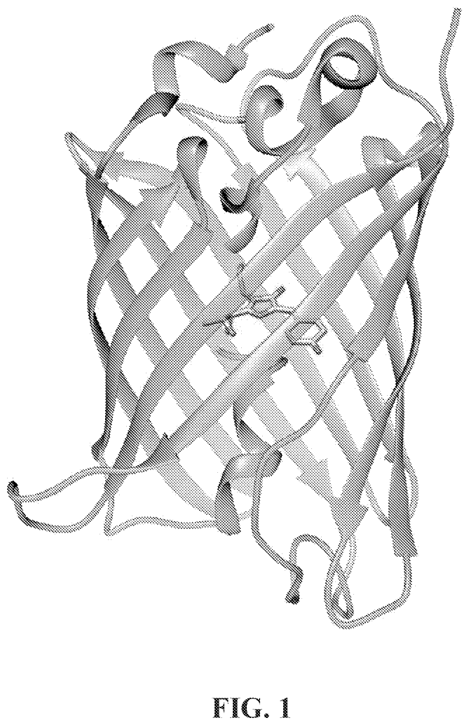

Leave-one-out split green fluorescent protein systems (LOO_GFP systems) utilize protein-fragment complementation assays to link analyte binding with signal generation by GFP. The GFP chromophore, which is generated by post-translational modification of three residues (see Crone, D. E. et al. “GFP-based biosensors,” State of the Art in Biosensors—General Aspects 2013), resides at the center of an 11 β-strand barrel (FIG. 1). The LOO_GFP system may be engineered from superfolder OPT GFP, a variant of GFP optimized for higher solubility. Alternatively, the LOO_GFP system may be engineered from GFP or other known GFP variants.

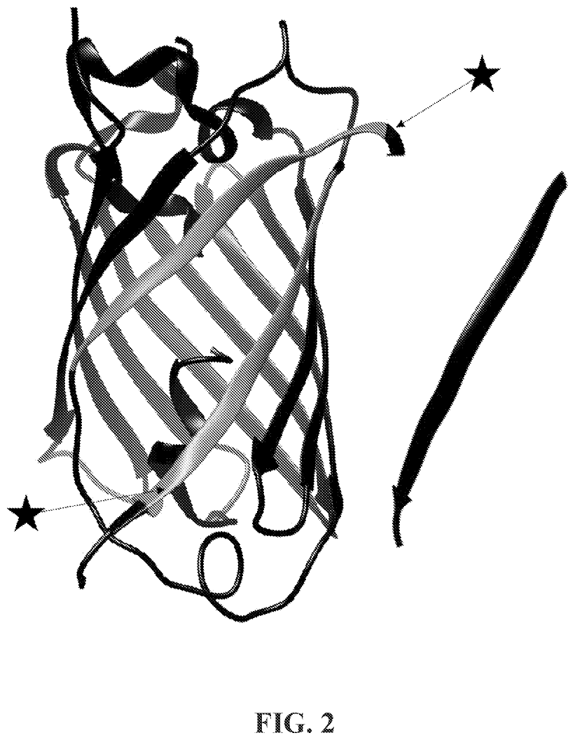

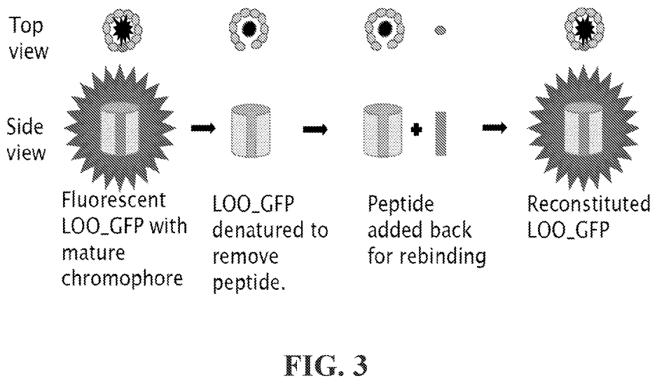

In LOO_GFPs, the protein sequence may be circularly permuted and truncated, allowing one or more of the 11 β-strand segments to be omitted while retaining solubility (FIG. 2). When a strand is removed, the chromophore is free to internally rotate such that the chromophore does not emit light. Binding by the excluded GFP peptide completes the protein structure, immobilizes the chromophore, and restores fluorescence. The excluded portion thus becomes the peptide analyte, and upon binding to LOO_GFP, creates a fluorescent sensor. This approach unites sensing and reporting in a single molecule. Such process is shown in FIG. 3.

Because only a small segment is left out, most of the folding pathway is retained and the unbound LOO_GFP may fold to a near-native structure, ready to bind the peptide and complete the formation of the β barrel. This strategy promotes proper folding and chromophore maturation, which can require several hours. The LOO_GFP and the left-out peptide are either co-expressed or pre-incubated together and thus the “priming” peptide must be removed to empty the binding site. Thereafter, addition of the target peptide reconstitutes the complete protein structure and chromophore fluorescence (FIG. 3). LOO_GFP variants are typically named after the removed strand. For instance, in LOO7 the 7th strand is removed and the corresponding peptide, s7, would function as the analyte. For purposes of this disclosure LOO7 (or LOO #) is used interchangeably with LOO7_GFP (or LOO #_GFP).

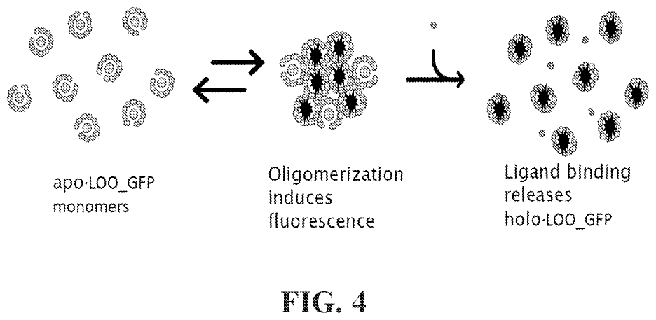

While LOO_GFP systems are promising self-reporting biosensors, such systems suffer from high background fluorescence and reduced signal-to-noise ratio. For example, in previous work, LOO7 and a designed influenza biosensor, LOO7-HA4, were found to form oligomers of variable size in the unbound state (FIG. 4), which creates background LOO7 and LOO7-HA4 fluorescence in the absence of either target peptide (see Huang, Y.-ming et al. “Toward computationally designed self-reporting biosensors using leave-one-out green fluorescent protein,” Biochemistry 2015, 54 (40), 6263-6273). This oligomerization acts as a nonspecific competitor, and thus alters the noise/minimal detectable level of the LOO7 platform (see Hassibi, A. et al. “On noise processes and limits of performance in Biosensors,” Journal of Applied Physics 2007, 102 (1), 014909). This problem has been reported for other GFP-based biosensors as well (see Romei, M. G., Boxer, S. G. “Split Green Fluorescent Proteins: Scope, limitations, and outlook,” Annual Review of Biophysics 2019, 48 (1) 19-44).

The effects of non-specific binding can generally be reduced by raising the concentration of the biosensor and target analyte relative to the non-specific binder. However, in the case of a LOO_GFP system, this problem is not easily solved by reducing the concentration of the biosensor and target analyte relative to the non-specific binder because, in the case of oligomerization, the non-specific binder is the biosensor. Moreover, split fluorescent proteins typically have a lower quantum yield than the original protein due to a less stable β-barrel structure, reducing the signal even further (see Köker, T. et al. “Characterization of split fluorescent protein variants and quantitative analyses of their self-assembly process,” Scientific Reports 2018, 8 (1)). In the case of LOO7, the quantum yield for s7: LOO7 was 40% that of the parent protein OPT_GFP8 (see Huang, Y.-ming et al. “Toward computationally designed self-reporting biosensors using leave-one-out green fluorescent protein,” Biochemistry 2015, 54 (40), 6263-6273).

Accordingly, there remains a need for LOO_GFP systems with improved dynamic range, i.e., the maximum signal minus the signal from the background or noise.

### LOO_GFP-Ubx Systems

The present disclosure describes embodiments of an improved LOO_GFP system and methods of making and using thereof. We have surprisingly found that immobilization of LOO_GFP within protein materials, for example Drosophila melanogaster Ultrabithorax (Ubx) (i.e., a “LOO_GFP-Ubx” system), provide an opportunity to both sterically block non-specific LOO_GFP oligomerization and stabilize the LOO_GFPs by effectively encasing the LOO_GFP cargo protein at high density within the materials matrix. As a result, LOO_GFP-Ubx systems provide improved biosensor function, increasing the fluorescent signal, preventing LOO_GFP oligomerization (thus reducing associated background fluorescence), and increasing the stability and thus lifetime of the biosensor, potentially enabling the biosensor to be deployed without maintaining a cold chain for storage. By both increasing the signal and decreasing the background, immobilization of LOO_GFP in Ubx biomaterials has increased the dynamic range of the LOO_GFP biosensor.

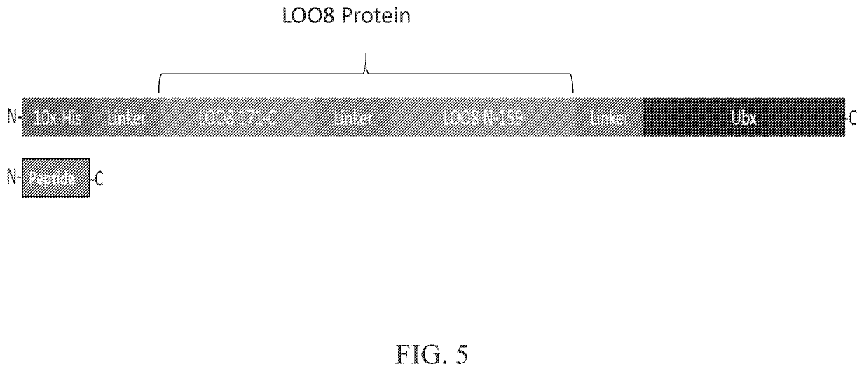

LOO_GFP systems may be formed according to example methods described in greater detail below. In preferred embodiments, the LOO_GFP is formed from a superfolder OPT GFP with the eighth β-strand omitted, referred to for purposes of this disclosure as LOO8 or LOO8_GFP. LOO8_GFP has high solubility and expresses well when genetically fused to the materials-forming protein, Ubx. However, it is to be understood that any suitable β-strand may be omitted to create LOO_GFP variants. For example, strand 11 may also be omitted. For purposes of this disclosure, an omitted β-strand may alternatively be referred to as sβ, where β denotes the nth strand removed. In preferred embodiments, binding by a synthetic peptide having an identical amino acid sequence as the omitted β-strand may complete the protein structure, immobilize the chromophore, and restore fluorescence. However, it is to be understood that LOO_GFP may be re-designed to bind and detect peptides having sequences different from that of the omitted β-strand. LOO_GFP-Ubx systems may be formed according to example methods described in greater detail below. In mild buffers, Ubx self-assembles into robust, biocompatible, non-immunogenic materials at the air-water interface. The resulting materials are stabilized by spontaneously forming dityrosine bonds. Functional proteins can be incorporated into Ubx materials through gene fusion. In this approach, adding the gene encoding the functional protein adjacent to the gene encoding Ubx with no intervening stop codons will result in a fusion gene. This fusion gene produces a single polypeptide harboring the sequences of both the functional protein and Ubx (FIG. 5). This approach provides a very high concentration of functional protein in the materials, resulting in a very strong fluorescent signal. Thus far, all Ubx fusion proteins tested are able to both self-assemble into materials and retain the function of the appended protein (see Huang, Z. et al. “Functionalization and patterning of protein-based materials using active Ultrabithorax chimeras,” Advanced Functional Materials 2011, 21 (14), 2633-2640; Howell, D. W. et al. “Identification of multiple dityrosine bonds in materials composed of the drosophila protein ultrabithorax,” Advanced Functional Materials 2015, 25 (37), 5988-5998; Tsai, S.-P. et al. “The effect of protein fusions on the production and mechanical properties of protein-based materials,” Advanced Functional Materials 2015, 25 (9), 1442-1450). Assembly of Ubx biomaterials was previously described in U.S. Pat. No. 11,059,872, which is herein incorporated by reference.

In some embodiments, a gene encoding LOO_GFP may be fused to a gene encoding Ubx to produce a single polypeptide containing the sequences of both functional protein LOO_GFP and Ubx, referred to as LOO_GFP-Ubx for purposes of this disclosure. LOO_GFP-Ubx may be co-expressed with its corresponding omitted β-strand (sβ) to complete the GFP structure and enable chromophore maturation. The resulting complex may be purified and assembled into fibers. Notably, LOO_GFP-Ubx may exhibit increased fluorescent signal relative to LOO_GFP and may additionally or alternatively exhibit increased stability relative to LOO_GFP.

For example, as shown in FIG. 5, in preferred embodiments a gene encoding LOO8_GFP (see, e.g., SEQ ID NO. 1) may be fused to a gene encoding Ubx to produce a single LOO8_GFP-Ubx fusion protein (see, e.g., SEQ ID NO. 2). In preferred embodiments, the LOO8_GFP has at least least 90% homology to SEQ ID NO. 1, at least 95% homology to SEQ ID NO. 1, or 100% homology to SEQ ID NO. 1. LOO8_GFP-Ubx fusion protein may include an N-terminal His tag, a LOO8 protein, Ubx, and GS linkers. The LOO8_GFP protein may comprise amino acids 172-238, an artificial linker (GGTGGS), and amino acids 1-158 of OPT GFP. In preferred embodiments, the LOO8_GFP-Ubx fusion protein has at least 90% homology to SEQ ID NO. 2, at least 95% homology to SEQ ID NO. 2, or 100% homology to SEQ ID NO. 2. An untagged s8 peptide corresponding to OPT GFP residues 159-171 (see, e.g., SEQ ID NO. 3) may be co-expressed with LOO8_GFP-Ubx. Preferably, the s8 peptide has at least 90% homology to SEQ ID NO. 3, at least 95% homology to SEQ ID NO. 3, or 100% homology to SEQ ID NO. 3. Expression tests of LOO7_GFP-, LOO8_GFP-, and LOO9_GFP-Ubx found that LOO8_GFP-Ubx had the best expression in E. coli. Therefore, the production of Ubx fusion monomers is sensitive to the protein being appended, even when comparing different LOO proteins, which have nearly the same composition as each other and their parent protein.

Expression tests have also shown that LOO11_GFP-Ubx has a high level of expression in E. coli. A gene encoding LOO11_GFP (see, e.g., SEQ ID NO. 4) may be fused to a gene encoding Ubx to produce a single LOO11_GFP-Ubx fusion protein (see, e.g., SEQ ID NO. 5). In preferred embodiments, the LOO11_GFP has at least least 90% homology to SEQ ID NO. 4, at least 95% homology to SEQ ID NO. 4, or 100% homology to SEQ ID NO. 4. Further, it is preferable that the LOO11_GFP-Ubx fusion protein has at least 90% homology to SEQ ID NO. 5, at least 95% homology to SEQ ID NO. 5, or 100% homology to SEQ ID NO. 5. An untagged s11 peptide (see, e.g., SEQ ID NO. 6) may be co-expressed with LOO11_GFP-Ubx. Preferably, the s11 peptide has at least 90% homology to SEQ ID NO. 6, at least 95% homology to SEQ ID NO. 6, or 100% homology to SEQ ID NO. 6.

Because LOO_GFP-Ubx and Ubx self-assemble at the same rate, mixing soluble LOO_GFP-Ubx and plain Ubx monomers in a specific ratio may produce fibers comprising LOO_GFP-Ubx and Ubx in the same ratio. Such composition may prevent the oligomerization of fluorescent, unbound LOO_GFP oligomers, reducing background noise relative to fibers comprising 100% LOO_GFP-Ubx. Thus, in some embodiments, fibers may comprise varying ratios of LOO_GFP-Ubx and Ubx. In preferred embodiments, fibers may comprise approximately 10% LOO8-Ubx and approximately 90% Ubx.

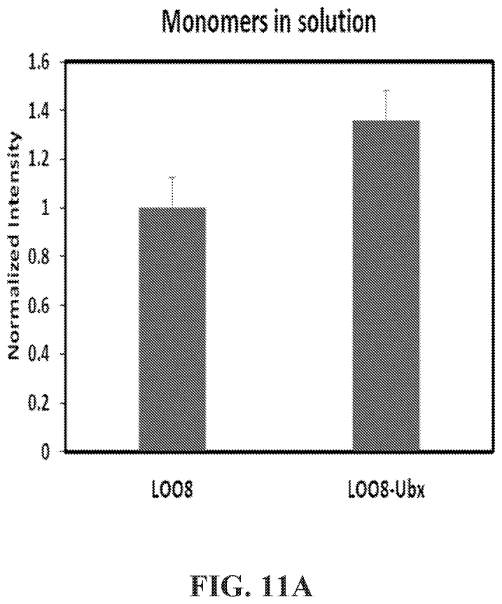

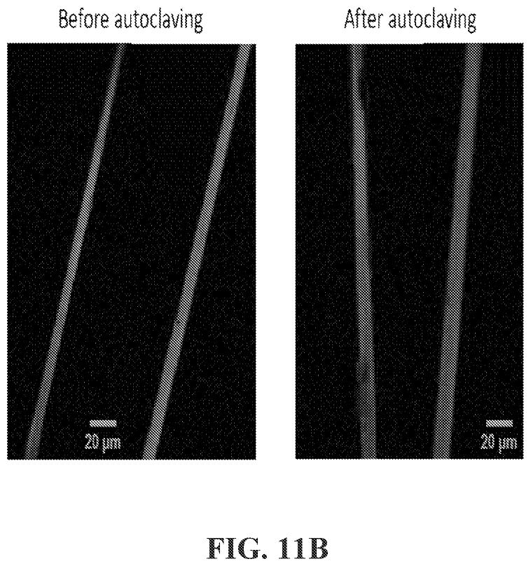

Additionally, LOO_GFP-Ubx monomers may exhibit significantly increased fluorescent signal compared to LOO_GFP monomers. In preferred embodiments, LOO8-Ubx may exhibit significantly increased fluorescent signal compared to LOO8 monomers (FIG. 11A). Thus, LOO_GFP-Ubx may have significantly higher signal-to-noise ratio than monomeric LOO_GFP.

In some embodiments, LOO_GFP-Ubx may be denatured to remove sβ, producing fibers capable of being used as a biosensor. Any suitable method for removing sβ may be used. In some embodiments, methods of denaturing and reconstituting fluorescence may include: denaturing LOO_GFP-Ubx with a denaturing agent, which in some embodiments may additionally comprise a salt and/or a detergent; washing with a water-based salt solution, which in some embodiments may additionally comprise a salt and/or detergent; and incubating with synthetic sβ peptide. An ordinary artisan may appreciate that washing of LOO_GFP-Ubx may be repeated as necessary. In some embodiments, methods of denaturing LOO_GFP-Ubx may include optimizing the denaturing buffer, incubation time, and fiber diameter to reduce background fluorescence due to oligomerization and optimize fluorescence recovery. An ordinary artisan may appreciate that signal and background levels in LOO_GFP-Ubx systems may be influenced by the fiber diameter, percent of fused monomers, and buffer conditions.

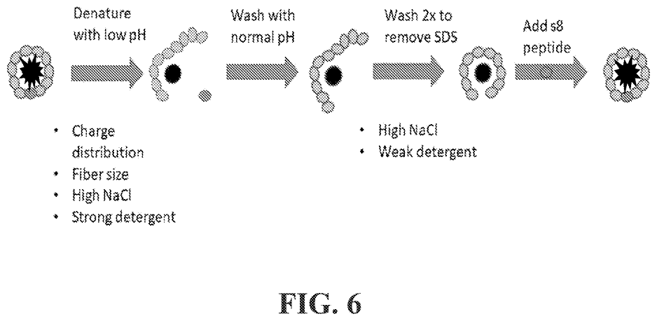

For example, as shown in FIG. 6, preferred methods for removing and reconstituting fluorescence of an example LOO8-Ubx biosensor may include, for approximately 10% LOO8_GFP-Ubx materials having approximately 10-20 μm diameters, (1) denaturing with a denaturing buffer having a pH of approximately 2, comprising approximately 1.25 M NaCl and approximately 0.1% SDS; (2) washing with phosphate buffered saline (PBS), raising the pH to approximately 8.0; (3) washing twice with approximately 1.25 M NaCl and approximately 0.1% 3-[(3-cholamidopropyl)dimethylammonio]-1-propanesulfonate (CHAPS, cat #C3023-1G), and (4) incubating with synthetic s8 to recover fluorescence.

Fusing LOO8_GFP and its noncovalently bound peptide (s8) to self-assembling Ubx is the first time a heterodimer has been incorporated into protein biomaterials via gene fusion and represents a notable finding for future functionalization of protein materials. The ability to incorporate heterodimers into protein materials via gene fusion greatly expands the potential of this technique, since many proteins that are desirable to incorporate into materials, including enzymes and growth factors, may be heterodimers. Because both the material and biosensor are produced as a single molecule, the cost and time required for multiple preparations and tethering the biosensor is saved. Unlike prior non-covalent attachment of LOO_GFP biosensors to resin, the covalent attachment provided by the gene fusion technique is expected to substantially reduce sensor leaching.

Some specific example embodiments of the invention may be understood by reference to one or more of the examples provided herein.

### EXAMPLES

### Example 1. Plasmid Construction of LOO8 and Ubx

pCDFDuet-1-LOO8-GFP plasmid were used as the parent vector to co-express LOO8_GFP-Ubx and its left-out peptide (s8). The two protein sequences were inserted at sites termed “MCS1” and “MCS2”, respectively. Ubx splicing isoform Ia (GenBank AAN13718.1) was inserted between the BamHI and EcoRI sites in the MCS1 site. MCS2 contains the left-out peptide “NGIKANFTVRHNV,” termed “priming s8”, fused C-terminally to the carrier protein Ssp-DnaB mini-intein by insertion into the BglII and EcoRV sites, as previously described (see Huang, Y.-M. et al. “Quantitative in vivo solubility and reconstitution of truncated circular permutants of green fluorescent protein,” Protein Science 2011, 20 (11), 1775-1780). Thus s8 is produced as a fusion protein and subsequently cleaved to create free s8 peptide.

### Example 2. Plasmid Construction of EGFP and Ubx

pET19b-UbxIa and pET19b-EGFP-UbxIa plasmid were also used. EGFP (enhanced green fluorescent protein) is a variant of GFP that has a greater fluorescent intensity and higher folding efficiency than wild-type GFP. As previously described, this construct contains the Ubx mRNA splicing isoform Ia (GenBank AAN13718.1) inserted between the NdeI and BamHI sites of pET19b vector (Novagen), with EGFP inserted in the NdeI site between the N-terminal His-tag and Ubx (see Tsai, S.-P. et al. “The effect of protein fusions on the production and mechanical properties of protein-based materials,” Advanced Functional Materials 2015, 25 (9), 1442-1450). EGFP and Ubx are separated by a short linker sequence “GSGSHMGSGS” to allow proper folding.

### Example 3. Expression and Purification of LOO8-Ubx

Protocols were used as established for expression and purification of Ubx and EGFP-Ubx protein fusions into materials (see Greer, A. M. et al. “The drosophila transcription factor ultrabithorax self-assembles into protein-based biomaterials with multiple morphologies,” Biomacromolecules 2009, 10 (4), 829-837). To express LOO8_GFP-Ubx and its cognate pCDFDuet-1-LOO8-GFP peptide, the plasmid was transformed into Rosetta (DE3) pLysS cells (Novagen). Single colonies were used to inoculate overnight liquid cultures and 10 mL of the overnight culture was used to inoculate a 1 L LB culture. Both cultures contained 0.03 mg/L chloramphenicol and 0.03 mg/L streptomycin. Protein expression was induced at mid-log phase (OD₆₀₀=0.6) with 1×10⁻³ M isopropyl-β-D-1-thiogalactopyranoside (IPTG) for 5 h, then cells were harvested by centrifugation at 7000 g for 10 min at 4° C. and stored at −20° C. Frozen cell pellets corresponding to 2 L of cell culture were lysed according to established protocols in a lysis buffer containing 50 mM NaH₂PO₄, 500 mM NaCl, 5% glucose, 20 mM imidazole, 1.5 mM PMSF, 5 mM DTT, 0.2 mg/mL lysozyme, Complete Proteinase Inhibitor Mixture (Roche), and 0.04 mg/mL DNase I at pH 8.75 (see Greer, A. M. et al. “The drosophila transcription factor ultrabithorax self-assembles into protein-based biomaterials with multiple morphologies,” Biomacromolecules 2009, 10 (4), 829-837). Cell debris was removed by centrifugation at 18000 g for 30 min at 4° C. Protein was purified from the clarified cell lysate using 5 mL HisPur™ Ni-NTA Superflow Agarose (ThermoFisher Scientific), which was pre-equilibrated with 2 column volumes of wash buffer (50 mM NaH₂PO₄, 500 mM NaCl, and 5% glucose at pH 8.0). Bound resin was washed with 1 column volume of wash buffer and 1 column volume of wash buffer containing 20 mM imidazole. Protein was eluted using a buffer containing 50 mM NaH₂PO₄, 500 mM NaCl, 5% glucose, and 300 mM imidazole at pH 7.0. The concentration of purified protein in samples was determined using the BioRad protein assay (BioRad). Fibers were pulled from films produced in a “buffer reservoir” with 1-2 mg of protein per reservoir, which contained 250 mL of the wash buffer described above. After a 10-minute incubation, films were compressed by drawing a plastic bar across the surface of the buffer, then fibers were pulled from the films and wrapped around 5 mm sterile plastic inoculation loops (hereafter “loops”). Fibers on loops were allowed to air-dry and stored upright in air until use, typically within 1-3 days. To prevent LOO8_GFP oligomerization, all fibers used for priming, s8 removal, and rebinding experiments were composed of 10% LOO8_GFP-Ubx and 90% Ubx, unless otherwise noted. Creating fibers with defined mixtures of proteins is possible because EGFP-Ubx and plain Ubx self-assemble equally well.

### Example 4. Characterization of LOO8-Ubx using Spectroscopy and Confocal Imaging

Spectroscopic measurements: For emission and excitation spectra, fluorescence spectroscopy was performed using a JASCO-815 fluorometer and a 10-mm pathlength quartz cuvette. Proteins were diluted into wash buffer, described above, to a concentration of 5 nM. Excitation spectra read at 490 nm and emission spectra read at 510 nm were monitored at 23° C. using a bandwidth of 1 nm for samples in solution and the maximum peak intensity was defined as 1. For fluorescent intensity measurements of LOO8_GFP and LOO8_GFP-Ubx monomers, samples were diluted in water to 100 nM and excited at 490 nm. The emission spectra were collected with a slit width of 10 nm at 23° C. and the maximum peak intensity was defined as 1.

Confocal images and fluorescent intensity measurements: For imaging, loops with fibers were transferred from the 24-well plate to a glass slide or coverslip and kept hydrated with a drop of distilled water. Fibers were imaged using confocal microscopy on a Nikon Eclipse Ti A1R equipped with NIS Elements AR software to assess fiber diameter and for fluorescent intensity analysis. Images were captured using a 20× objective and each experiment used untreated fibers as controls for fluorescent intensity. Fluorescent intensity of LOO8_GFP-Ubx was normalized using fiber diameter and dityrosine fluorescence, measured in the 4′,6-diamidino-2-phenylindole (DAPI) channel, which accounts for the amount of materials present. For each fiber image, background is defined as the detected light originating outside the resolution volume (i.e., a fiber) and signal is defined as the detected light originating within the resolution volume. For measurements of non-fluorescent fibers, the fluorescent intensity, or signal, is below the background of the microscope due to a lack of light scattering within the fiber.

### Example 5. Verification of LOO8-Ubx Materials Structure and Function

Protein complementation assays based on GFP provide a convenient mechanism to unite analyte detection and signal generation in a single molecule. However, the resulting LOO_GFP biosensor proteins can oligomerize and fluoresce in the absence of the target peptide, increasing the background signal (FIG. 4). Physically holding the LOO_GFP domains apart by covalently tethering them to a solid support both prevents oligomerization and immobilizes the LOO_GFP to facilitate separation during analyte binding and washing.

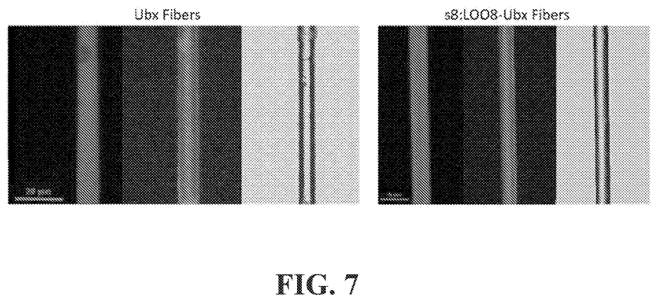

To address the issue of oligomerization for LOO8_GFP, LOO8_GFP-Ubx materials were created by fusing the LOO8_GFP gene to the Ubx gene (FIG. 5 and SEQ ID NO. 2) and co-expressed it with the left-out strand 8 priming peptide (s8, SEQ ID NO. 3) to complete the GFP structure and thus enable chromophore maturation. The resulting heterodimeric fusion protein complex (s8: LOO8_GFP-Ubx) was purified and assembled into fibers (FIG. 7). Because attaching LOO8_GFP to a self-assembling protein has the potential to alter the properties of one or both proteins, it is necessary to assess whether LOO8_GFP function alters the assembly or structure of the materials, as well as whether embedding LOO8_GFP in materials alters biosensor function.

### Example 5A. LOO8_GFP Fusion Does Not Damage Fiber Assembly

To test whether fusion to LOO8 damaged fiber assembly, we compared the assembly of LOO8_GFP-Ubx to that of plain Ubx and Enhanced Green Fluorescent Protein (EGFP)-Ubx. The LOO8_GFP-Ubx fusion protein successfully self-assembled into films that were then drawn into green fluorescent fibers. The timescale for materials assembly matched that for both plain Ubx materials and EGFP-Ubx materials. For the same protein concentrations, the size of films and thus the length and diameter of fibers composed of LOO8_GFP-Ubx is similar to that of Ubx and EGFP-Ubx, suggesting that the protein's assembly rate is unchanged, as previously demonstrated for Ubx and EGFP-Ubx (see Tsai, S.-P. et al., “The effect of protein fusions on the production and mechanical properties of protein-based materials,” Advanced Functional Materials 2015, 25 (9), 1442-1450).

Because conditions that damage Ubx self-assembly also harm fiber structure, the morphology of the fibers must also be examined. For instance, Ubx truncation mutants generate thin, weak and/or malformed fibers, and Ubx films drawn through organic solvents create misshapen fibers that vary in width and contain frays or pits. The morphology of the s8: LOO8_GFP-Ubx fibers was typical for Ubx materials as indicated by microscopy (FIG. 7). s8: LOO8_GFP-Ubx fibers have a uniform diameter, no pits or defects, and fluoresce due to dityrosine bond formation, similar to Ubx fibers and materials composed of other Ubx fusion proteins. The normal appearance of these fibers and their ability to self-assemble on a normal timescale further suggests that fusion to LOO8_GFP did not impact Ubx assembly.

### Example 5B. LOO8_GFP Fusion Does Not Alter LOO8_GFP Function

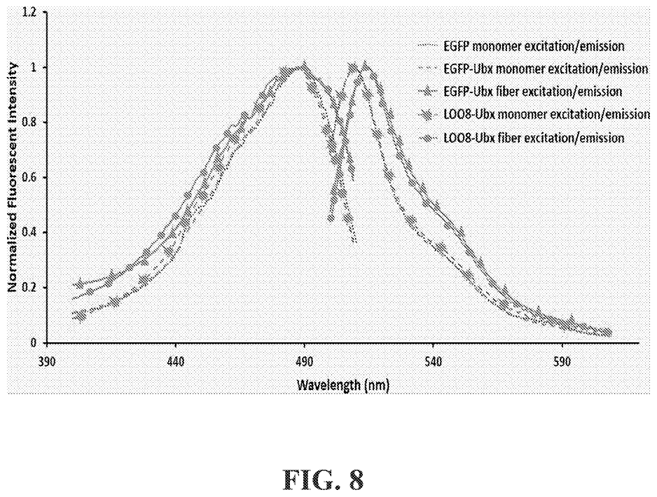

Conversely, fusion to Ubx and assembly into materials could impact LOO8_GFP function. Changes in LOO_GFP protein structure can alter the chemical environment and/or dynamics of the fluorophore, changing the shape or position of its emission spectrum. To determine whether Ubx fusion and materials assembly alters LOO8_GFP function, the fluorescence spectra of s8: LOO8_GFP-Ubx monomers was compared to that of EGFP monomers. EGFP is very stable and is known to retain activity in Ubx monomers and fibers. Purified s8: LOO8_GFP-Ubx soluble monomers fluoresced green, and the excitation and emission spectra of s8: LOO8_GFP-Ubx monomers matched that of EGFP and of EGFP-Ubx monomers (FIG. 8, dotted and dashed lines, including dashed lines with stars). Additionally, the excitation and emission spectra of fibers composed of s8: LOO8_GFP-Ubx matched those of fibers composed of EGFP-Ubx (FIG. 8, solid lines with triangles or circles).

Thus, immobilization in Ubx materials did not appear to alter the function of LOO8 relative to its more stable counterpart, EGFP. Interestingly, the fluorescence emission spectra of both s8: LOO8_GFP-Ubx and EGFP-Ubx in fiber form is red-shifted relative to the emission spectra of the corresponding monomers prior to polymerization (from 510 to 515). Because both EGFP and LOO8_GFP are affected, this red shift is not potentiated by the amino acid sequence alterations involved in creating LOO8_GFP. A likely explanation is that fiber formation places charged amino acids near the fluorescent proteins. A charged environment decreases the energy of the highly polarized excited state of the chromophore, red-shifting the emission spectrum. The largest density of charged residues in LOO8_GFP-Ubx is in the Ubx homeodomain, in which 17 of the 60 amino acids are lysine or arginine. Most of these positively charged amino acids are located in the disordered N-terminal arm and helix three of the homeodomain. Thus, one possibility is that the negatively charged residues on the surface of LOO8_GFP form ionic bonds with the positively charged residues in the Ubx homeodomain, leading to a red shift in fluorescence by the chromophore. This red-shift phenomenon has previously been observed for dityrosine bonds in Ubx, in which the blue fluorescence emission peak is shifted from the normal range of 410-430 nm to 438 nm. These data suggest that the ability of LOO8_GFP to fluoresce is not negatively affected by the fusion to Ubx or by the polymerization of Ubx.

### Example 6. Methods of Removing s8 peptide from LOO8_GFP-Ubx

Denaturing assay: To determine the effect of denaturing washes on the removal of bound priming s8, loops supporting 4-6 100% LOO8_GFP-Ubx fibers were placed in a 24-well plate and incubated in 1 mL of 0.1 M glycine-HCl buffer, pH 2.0 for 5 seconds or 4.5 M GdnHCl (i.e., guanidinium chloride) for 15 minutes. Fibers were rinsed by quickly submerging the loops in 2 different wells, each containing 1 mL of PBS (20 mM NaH₂PO₄, 80 mM Na₂HPO₄, 500 mM NaCl, pH 8.0), then submerging the loop in PBS for 10 minutes. After the final wash, fibers were imaged as described above.

Denaturing wash time assay: To test the time required to remove bound priming s8, loops supporting 4-6 10% LOO8_GFP-Ubx fibers were placed in a 24-well plate and incubated in 1 mL of 0.1 M glycine-HCl buffer, pH 2.0 for 0.5, 1, 5, 10, 20, or 30 minutes. Fibers were rinsed by successively placing the loops in 3 different wells, each containing 1 mL of PBS (20 mM NaH₂PO₄, 80 mM Na₂HPO₄, 500 mM NaCl, pH 8.0) for 10 minutes each. After the final wash, fibers were imaged as described above.

### Example 7. Elimination of Background Fluorescence in LOO8_GFP Biosensors

To use these sensors, we must first establish conditions that produce as low a baseline signal as possible. The left-out peptide s8 is co-expressed with LOO8_GFP-Ubx to reconstitute protein folding and allow chromophore maturation in vivo. Naturally, priming s8 must be removed from the fibers by unfolding/refolding before the fibers can be used as a biosensor, producing apoLOO8_GFP-Ubx. There are many methods for removing priming s8. An ideal method would remove all of the s8 peptide, creating the lowest possible baseline signal, without compromising the LOO8_GFP protein and reducing the maximum obtainable signal.

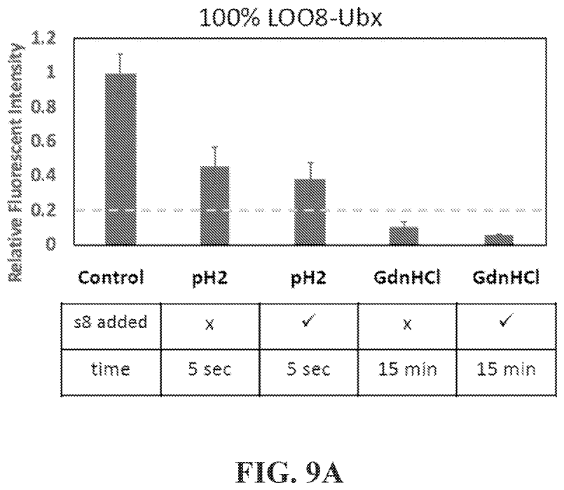

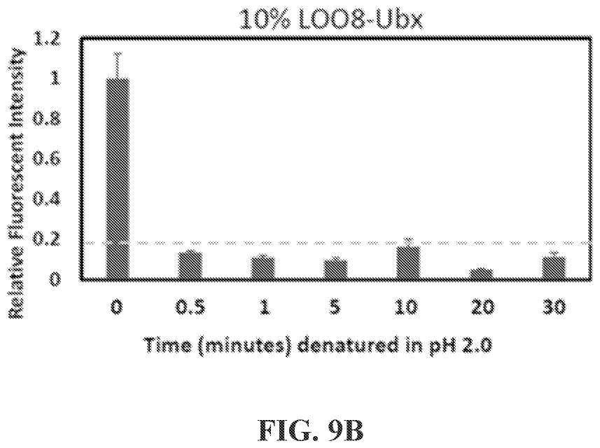

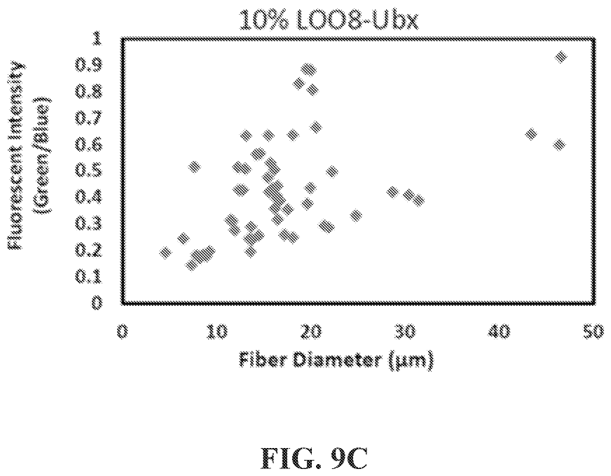

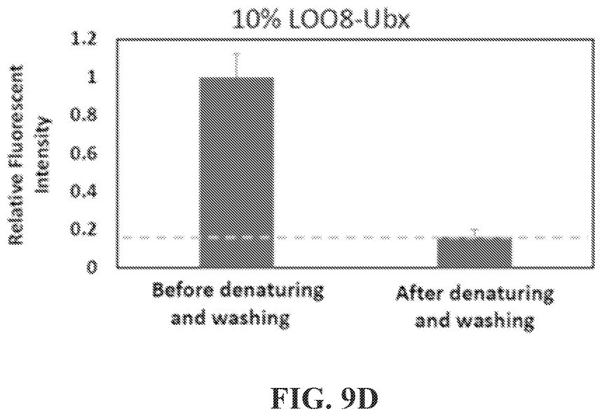

An initial test used a denaturing buffer at pH 2 for 5 seconds to loosen s8: LOO8_GFP and allow priming s8 to escape the fiber. This low pH was previously used to unfold LOO7 monomers (see Huang, Y.-ming et al. “Toward computationally designed self-reporting biosensors using leave-one-out green fluorescent protein,” Biochemistry 2015, 54 (40), 6263-6273). When fibers composed of 100% LOO8_GFP-Ubx were exposed to this buffer, they immediately lost fluorescence. However, after washing and returning the fibers to neutral pH, a substantial amount of fluorescence remained in samples containing only the LOO8_GFP-Ubx fiber as well as samples in which synthetic s8 had been added back to the LOO8_GFP-Ubx fiber (FIG. 9A). This data suggests that either (i) the LOO_GFPs are still oligomerizing, (ii) priming s8 is not being released from the LOO8_GFP protein, or (iii) that priming s8 is not able to diffuse out of the materials in that time and was rebinding to refolded LOO8_GFP.

### Example 7A. Effect of LOO8_GFP-Ubx to Ubx Ratio on Background Fluorescence

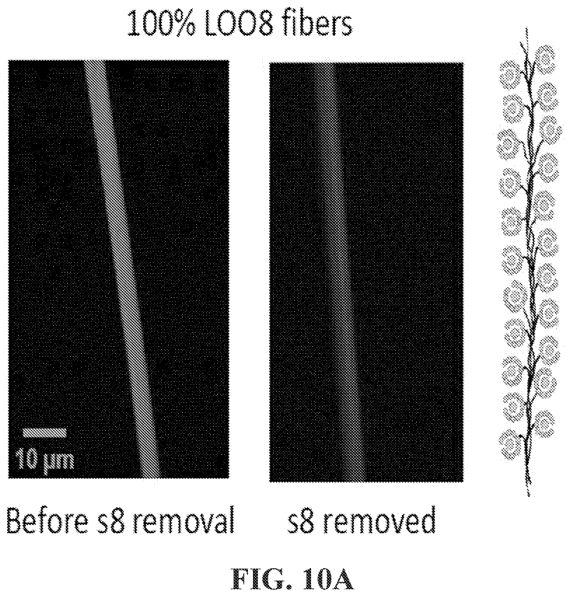

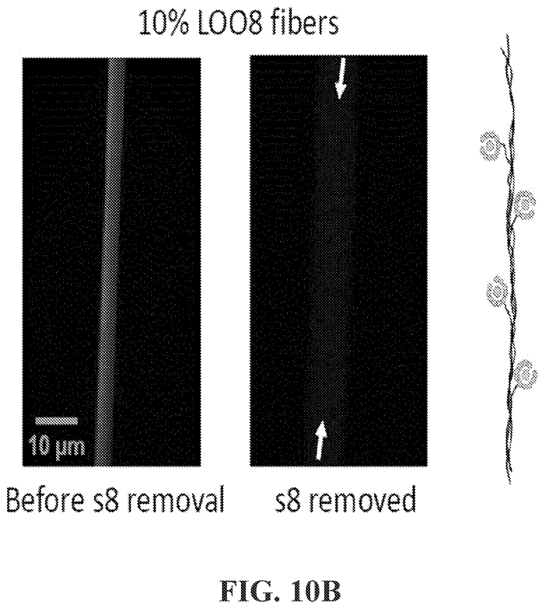

The potential for LOO8_GFP oligomerization to contribute to the background fluorescence is discussed herein. In a previous study of soluble LOO7, the intensity of background fluorescence caused by apoLOO7 oligomerization was so severe that it rivaled that of the holoLOO7 peptide complex (see Huang, Y.-ming et al. “Toward computationally designed self-reporting biosensors using leave-one-out green fluorescent protein,” Biochemistry 2015, 54 (40), 6263-6273). The following study helped to determine whether immobilizing LOO8_GFP in Ubx materials can prevent LOO8_GFP oligomerization and the resulting increase in background fluorescence. For initial studies, materials composed of 100% LOO8_GFP-Ubx were used (FIG. 10A). The extent of fluorescence in 100% LOO8_GFP-Ubx fibers after priming s8 removal was significantly reduced compared to before s8 removal, suggesting LOO8_GFP-Ubx fibers have less oligomerization-associated fluorescence than soluble LOO7. This effect could be due to using LOO8_GFP, a more soluble monomer, or to immobilizing LOO8_GFP in Ubx materials.

Despite this improvement, some fluorescence remained in apoLOO8_GFP-Ubx fibers indicating that oligomerization may still occur. To further reduce LOO8_GFP oligomerization, we also created materials composed of 10% LOO8_GFP-Ubx and 90% Ubx (FIG. 10B). Quantitatively reducing the concentration of the cargo LOO8_GFP protein is possible in LOO8_GFP-Ubx materials because GFP-Ubx and plain Ubx self-assemble at the same rate. Thus, mixing soluble GFP-Ubx and plain Ubx monomers in a specific ratio will produce fibers composed of these proteins in the same ratio. As expected, the 10:90 mixture produced morphologically normal fibers (FIG. 10B). Theoretically, at this ratio each LOO8_GFP will be entirely surrounded by Ubx. Without access to other LOO8s, there can be no oligomerization and thus no background fluorescence. In practice, these fibers ranged from displaying no background fluorescence to exhibiting some residual fluorescence after priming s8 removal. To achieve consistent results, other possible sources of background fluorescence must be eliminated.

### Example 7B. Effect of Denaturing Conditions on Background Fluorescence

A second possible source of background fluorescence is that the priming peptide s8 is not being released from LOO8_GFP, resulting in residual background fluorescence. Since incubating the fibers for 5 seconds at pH 2.0 failed to remove all background fluorescence, both the strength of the denaturant and the incubation time were increased. However, exposure of the fibers to 4.5 M GdnHCl, which for many proteins acts as a stronger denaturant than acid, for 15 minutes appeared to damage the LOO8 protein and prevent rebinding, as no signal was ever recovered above background after subsequently adding synthetic s8 (FIG. 9A). Previous work suggested proline isomerization or other slow changes in the unfolded state of GFP could lead to a “trapped” non-fluorescent state (see Rosenman, D. J. et al. “Green-lighting green fluorescent protein: Faster and more efficient folding by eliminating a cis-trans peptide isomerization event. Protein Science 2014, 23 (4), 400-410).

Since LOO8_GFP does not need to be completely denatured, only sufficiently loosened in structure to dislodge the priming s8 peptide, gentler acid denaturation techniques were used to avoid any slow conformational drift made possible by occupying the denatured state, but with an increased denaturation time to allow priming s8 to dissociate from LOO8_GFP. For all time points tested (30 seconds to 30 minutes), no residual fluorescence was detected above background levels for the microscope (FIG. 9B). If conformational drift and/or proline isomerization is present, then a shorter incubation time in denaturing conditions would favor refolding. Since removal of fluorescence is achieved quickly and remained essentially the same for all time points, a standard 10-minute denaturation time was adopted for all subsequent experiments to allow sufficient time for priming s8 to diffuse out of the fibers while minimizing putative conformational drift, thus retaining the ability to recover fluorescence. This approach allowed synthetic s8 to rebind LOO8_GFP and generate a fluorescent signal, as discussed in greater detail below.

### Example 7C. Effect of LOO8_GFP-Ubx Fiber Size on Background Fluorescence

The third possible source of background fluorescence is that priming s8 fails to escape materials and rebinds after the denaturation step. Larger diameter fibers will require more time for priming s8 to diffuse out of the fibers and for analyte to diffuse into the fibers. Thus, diameter may affect both the observed background and reconstituted fluorescence measurements. We asked whether the fluorescence remaining after denaturing and washing is dependent on fiber diameter.

LOO8_GFP-Ubx fibers of differing diameters were achieved using the following methodology. Loops with 10% LOO8-Ubx fibers were placed in a 24-well plate and denatured for 10 minutes to remove bound priming s8 with 1 mL of 0.1 M glycine-HCl buffer, pH 2.0. Fibers were rinsed by successively placing the loops in 3 different wells, each containing 1 mL of PBS (20 mM NaH₂PO₄, 80 mM Na₂HPO₄, 500 mM NaCl, pH 8.0) for 10 minutes each. After the final wash, fibers were imaged as described above and sorted based on the fiber diameter, measured using confocal microscopy.

Using fibers with diameters ranging from 4 to 50 μm (FIG. 9C), the only residual fluorescence observed was comparable to baseline. However, the raw green fluorescence data did reveal increased signal for larger fibers. To account for the differences in the size of the sample in the measurement for each fiber, the intensity of green fluorescence was divided by the intensity of blue dityrosine fluorescence, which reports on the amount of materials present. Even though the overall background levels are minimal, thicker fibers had a higher background fluorescence after being denatured for 10 minutes, and thinner fibers had a lower background fluorescence. Very thin fibers (<5 μm) are prone to breakage and are difficult to image; thus, fibers with diameters in the 10-20 μm range were chosen to minimize this effect in further experiments.

By 1) starting with a more soluble LOO8_GFP monomer, 2) reducing the concentration of LOO8_GFP proteins in Ubx materials, and 3) optimizing the denaturing buffer, incubation time, and fiber diameter, we can reliably eliminate the background fluorescence caused by oligomerization (FIG. 9D). This elimination of background dramatically improves the signal-to-noise ratio of the biosensor.

### Example 8. Enhancing LOO8_GFP-Ubx Signal

The other approach to increase the dynamic range of a biosensor is to maximize the detectable signal. In soluble LOO7 biosensors, the holoLOO7 peptide complex was only 40% as fluorescent as its parent protein, OPT GFP. The intensity of GFP fluorescence is inversely related to the dynamics of its chromophore, which, in turn, is dependent on GFP stability. Not surprisingly, removing a GFP strand and replacing it with a separate peptide destabilizes the protein, accounting for the reduction in fluorescence in the holoLOO7 peptide complex. Consequently, there is plenty of room to improve the amount of signal generated by LOO proteins.

### Example 8A. Ubx Fusion to LOO8_GFP Enhances LOO8 Fluorescence

Both fusion to Ubx and materials assembly have the potential to impact LOO_GFP stability, and thus LOO_GFP fluorescence. To determine if fusion to Ubx alters LOO8_GFP fluorescence, the relative fluorescence intensity of LOO8 was compared to that of LOO8_GFP-Ubx monomers, both at 5 nM. Surprisingly, Ubx fusion does enhance LOO8_GFP fluorescence: LOO8_GFP-Ubx was 1.4-fold as fluorescent as unfused LOO8_GFP (FIG. 11A). Given that LOO8_GFP is predicted to have a net negative charge, it is possible that intramolecular charge-charge interactions between the fluorescent proteins and the positively charged homeodomain enhance the stability, and hence the fluorescence, of these monomeric fusions. This hypothesis is supported by the prior observation of a higher breaking strain in EGFP-Ubx fibers (see Tsai, S.-P. et al. “The effect of protein fusions on the production and mechanical properties of protein-based materials,” Advanced Functional Materials 2015, 25 (9), 1442-1450), which was attributed to intermolecular EGFP: Ubx interactions, and in the red shift of the LOO8_GFP-Ubx fiber emission spectrum (FIG. 8). Another possibility is that the electrostatic interactions with Ubx stabilize the excited state of the chromophore, in which there is increased charge separation relative to the ground state. Such stabilization could increase the concentration of the excited state of LOO8_GFP-Ubx relative to monomeric LOO8 given the same 490 nm excitation, increasing quantum yield.

### Example 8B. Ubx Fusion to LOO8_GFP Enhances LOO8_GFP Stability

Ubx materials can stabilize other proteins to a surprising extent. Indeed, EGFP-Ubx fibers can be autoclaved or stored at room temperature for >9 years and still remain intact and green fluorescent (see Tsai, S.-P. et al. “The effect of protein fusions on the production and mechanical properties of protein-based materials,” Advanced Functional Materials 2015, 25 (9), 1442-1450). Stabilization of cargo proteins fused to protein-based materials has been observed for other materials as well and is generally attributed to confined geometry effects (see Zhang, Q. et. al. “Concerted asynchronous hula-twist photoisomerization in the S65T/H148D mutant of green fluorescent protein,” Angewandte Chemie International Edition 2014, 53 (33), 8649-8653). These experiments were repeated with s8: LOO8_GFP-Ubx and found that autoclaving LOO8_GFP-Ubx fibers did not significantly reduce the fluorescent signal (FIG. 11B). Fibers soaked in 100% ethanol for 30 minutes also remained fully fluorescent. These fluorescent data from monomers and fibers suggest a substantial increase in protein stability, which may further elevate the signal provided by LOO8_GFP when incorporated into Ubx materials.

### Example 8. Methods of Rebinding Synthetic s8 to LOO8_GFP-Ubx

Detergent assay: Synthetic s8 peptide “NGIKANFTVRHNV” (Genscript) was dissolved in 50 μl of dimethylformamide and diluted with PBS to a stock concentration of 0.12 mM, then aliquoted and stored at −80° C. until use. Loops with 10% LOO8_GFP-Ubx fibers were placed in a 24-well plate and denatured for 10 minutes to remove bound priming s8 with 1 mL of denaturing buffer (50 mM Tris pH 2.0, 50 mM NaH₂PO₄, 300 mM NaCl, 0.1% SDS, 100 mM BME). Fibers were rinsed by placing the loops in 1 mL of buffer 1 (50 mM Tris pH 8.75, 50 mM NaH₂PO₄, 300 mM NaCl) for 10 minutes, 1 mL of buffer 2 (50 mM Tris pH 7.5, 50 mM NaH₂PO₄, 300 mM NaCl, 10 mM imidazole, 0.1% CHAPS, 10 mM BME) for 10 minutes, and 1 mL of buffer 3 (50 mM Tris pH 7.5, 50 mM NaH₂PO₄, 300 mM NaCl, 20 mM imidazole, 0.1% CHAPS, 10 mM BME) for 10 minutes. Fibers were then incubated overnight either in PBS or 0.12 mM synthetic s8 (GenScript) diluted in PBS at 25° C. to bind synthetic s8 before imaging as described above.

NaCl concentration assay: Loops with 10% LOO8_GFP-Ubx fibers were placed in a 24-well plate and denatured for 10 minutes to remove bound priming s8 with 1 mL of 0.1 M glycine-HCl buffer, pH 2.0, containing elevated NaCl as indicated (0.5, 0.75, 1.25 or 1.5 M NaCl). Fibers were rinsed three times for 10 minutes each by placing the loops in different wells, each containing 1 mL PBS (20 mM NaH₂PO₄, 80 mM Na₂HPO₄, 500 mM NaCl, pH 8.0), supplemented with the same NaCl concentration used in the denaturing wash. Fibers were then incubated with either PBS or 0.12 mM synthetic s8 (GenScript) diluted in PBS and left overnight at 25° C. to bind synthetic s8 before imaging as described above.

Detergent and NaCl assay: To remove bound priming s8, loops with 10% LOO8_GFP-Ubx fibers were denatured for 10 minutes with 1 mL of high-salt denaturing buffer (50 mM Tris pH 2, 50 mM NaH₂PO₄, 1.25 M NaCl, 0.1% SDS, 100 mM BME) in a 24-well plate. Fibers were rinsed by submerging the loops in 1 mL of buffer 1 (50 mM Tris pH 8.75, 50 mM NaH₂PO₄, 300 mM NaCl) for 10 minutes, 1 mL of buffer 2 (50 mM Tris pH 7.5, 50 mM NaH₂PO₄, 1.25 M NaCl, 10 mM imidazole, 0.1% CHAPS, 10 mM BME) for 10 minutes, and 1 mL of buffer 3 (50 mM Tris pH 7.5, 50 mM NaH₂PO₄, 1.25 M NaCl, 20 mM imidazole, 0.1% CHAPS, 10 mM BME) for 10 minutes. Fibers were then placed either in PBS or 0.12 mM synthetic s8 (GenScript) diluted in PBS and left overnight at 25° C. to bind synthetic s8 before imaging as described above.

### Example 9. Effect of Rebinding Conditions on the Reconstitution of LOO8_GFP Fluorescence

The next challenge is to identify conditions in which synthetic s8 reliably rebinds the apoLOO8_GFP-Ubx fiber, displaying the fluorescent signal. An initial experiment using phosphate buffered saline (PBS) as the rebinding buffer did not reveal any synthetic s8 rebinding above background levels (FIG. 9A). In previous LOO7-GFP studies in which the protein was co-expressed with s7, the peptide was removed by lowering the pH to 2.0, adding SDS, then raising the pH to 8.0 and washing the SDS away using a weak detergent, CHAPS, all while non-covalently immobilized on a Ni-NTA agarose column. This method was found to recover more of the fluorescence than a simple pH jump, or by just adding and removing GdnHCl (see Huang, Y.-ming et al. “Complementation and reconstitution of fluorescence from circularly permuted and truncated green fluorescent protein,” Biochemistry 2009, 48 (5), 929-940; Huang, Y.-ming et al. “Toward computationally designed self-reporting biosensors using leave-one-out green fluorescent protein,” Biochemistry 2015, 54 (40), 6263-6273).

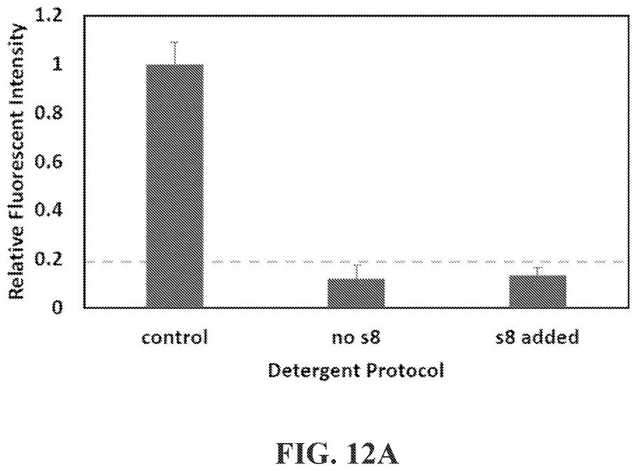

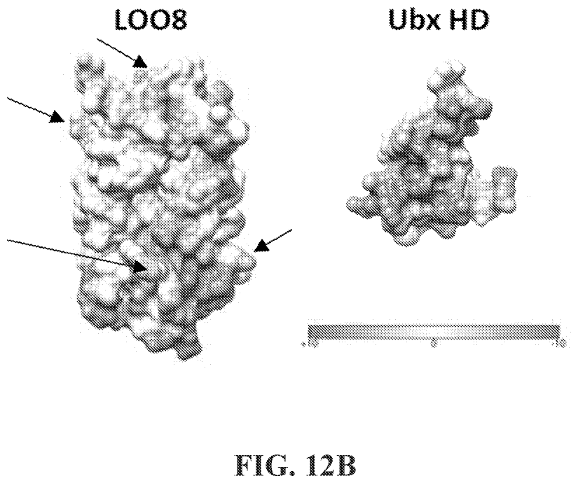

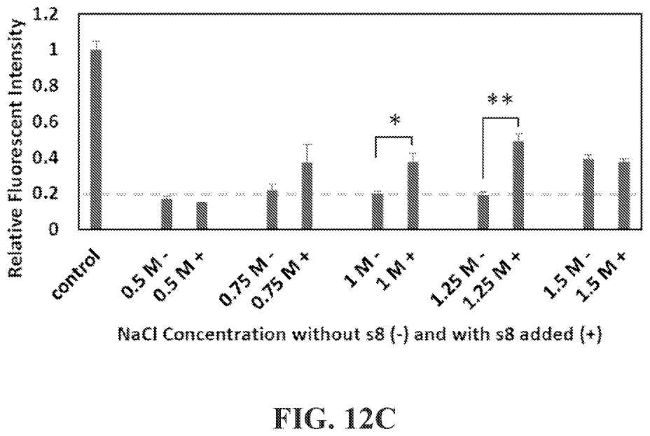

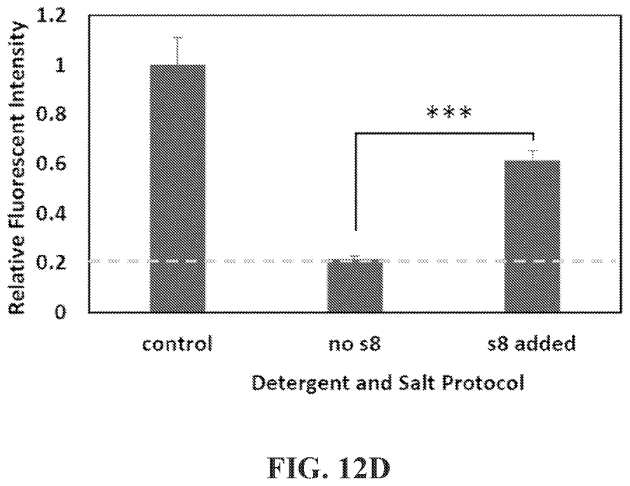

However, including SDS in the denaturing step and CHAPS in the washes did not result in any observable synthetic s8 rebinding (FIG. 12A). An important clue to the lack of s8 rebinding was provided by a prior study, in which ionic interactions between Ubx (predicted net charge +9) and EGFP (predicted net charge −2) were proposed to be the source of the increased strength of EGFP-Ubx fibers relative to fibers composed of only Ubx (see Tsai, S.-P. et al. “The effect of protein fusions on the production and mechanical properties of protein-based materials,” Advanced Functional Materials 2015, 25 (9), 1442-1450). Many of the negatively charged residues in apoLOO8_GFP surround the gap created when priming s8 is removed (FIG. 12B). It was hypothesized that in the absence of s8, the negatively charged face of apoLOO8_GFP binds Ubx, which then physically blocks synthetic s8 rebinding. Indeed, the red shift of the LOO8_GFP emission spectrum in fibers vs. monomers (FIG. 8) further suggests such interactions may be occurring. If true, increasing the salt concentration should reduce this effect.

### Example 9A. Use of NaCl in Denaturation and Wash Buffers Improves LOO8_GFP Fluorescence TAMU 6096

Synthetic s8 binding experiments were repeated with different concentrations of NaCl added to the denaturation and wash buffers in an attempt to screen charge interactions between LOO8 and Ubx and allow synthetic s8 to rebind and reconstitute fluorescence. For each NaCl concentration, samples in which synthetic s8 was added in the final step were compared to samples in which s8 was omitted to assess the levels of background fluorescence. Below 1 M NaCl, adding synthetic s8 did not recover significant fluorescence above background, suggesting this concentration was not sufficient to screen the charge interactions. At 1.5 M NaCl, the background is high and indistinguishable from the fluorescence recovered by adding synthetic s8, suggesting this very high salt concentration is prohibiting priming s8 from diffusing out of the fibers during the denaturation step. However, with 1 M, and 1.25 M NaCl concentrations, a significant increase in fluorescence over the background was observed when synthetic s8 is added (FIG. 12C).

### Example 9B. Use of NaCl and Detergents in Denaturation and Wash Buffers Improves LOO8_GFP Fluorescence

Using salt to screen charge interactions between LOO8_GFP and Ubx was then incorporated into the previously described protocol of adding detergents to the denaturation and wash steps to determine if recovery of fluorescence could be further improved. When 1.25 M NaCl was used in combination with the detergent, greater recovery of fluorescence was observed than when using 1.25 M NaCl alone (FIG. 12D). It is hypothesized that the addition of a detergent keeps LOO8 unfolded or loosely structured and may facilitate the diffusion of priming s8 out of and into the fibers, increasing the efficiency of the washes. Together, these results confirm that the LOO8_GFP biosensors function properly when immobilized within protein fibers.

The particular embodiments disclosed above are illustrative only, as the disclosure may be modified and practiced in different but equivalent manners apparent to those skilled in the art having the benefit of the teachings herein. For example, the process steps set forth above may be performed in a different order. Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

What is claimed is:

1. A composition comprising:
a. a protein-fragment complementation assay wherein at least one component is immobilized with ultrabiothorax (Ubx) biomaterials;
b, wherein the protein-fragment complementation assay comprises a fluorescent protein.

2. The composition of claim 1, wherein Ubx is fused to a component of the protein-fragment complementation assay.

3. The composition of claim 2, wherein the fluorescent protein is a split fluorescent protein system.

4. The composition of claim 3, wherein Ubx is fused to part of the split fluorescent protein system.

5. The composition of claim 1, 2, or 3, wherein the fluorescent protein is a green fluorescent protein or variant thereof.

6. The composition of claim 1, 2, or 3, wherein the fluorescent protein is OPT GPT.

7. The composition of claim 2, wherein Ubx is fused to a LOO_GFP.

8. The composition of claim 7, wherein Ubx is fused to a LOO8_GFP.

9. The composition of claim 7, wherein Ubx is fused to a LOO11_GFP.

10. A method of manufacturing LOO_GFP-Ubx suitable for a biosensor comprising:
a. co-expressing a LOO_GFP-Ubx fusion protein and a left-out peptide in a first host cell;
b. extracting protein from the first host cell;
c. incubating the extracted protein in a buffer reservoir to support film formation;
d. pulling fibers from the film;
e. air drying the fibers; and
f. washing the fibers with a denaturing wash to remove the left-out peptide.

11. The method of claim 10, wherein the LOO_GFP-Ubx is LOO8_GFP-Ubx and the left-out peptide is s8.

12. The method of claim 10, wherein the LOO_GFP-Ubx is LOO11_GFP-Ubx and the left-out peptide is s11.

13. The method of claim 10, wherein the LOO_GFP-Ubx fusion protein is expressed from a plasmid vector.

14. The method of claim 10, wherein the left-out peptide is expressed from a plasmid vector.

15. The method of claim 10, wherein the LOO_GFP-Ubx fusion protein and the left-out peptide are coexpressed from the same plasmid vector.

16. The method of claim 10, wherein the denaturing wash includes a detergent and a concentration of 1M NaCl to 1.25M NaCl.

17. The method of claim 10, further comprising:
(i) expressing Ubx in a second host cell; and
(ii) extracting Ubx protein from the second host cell,
wherein the incubating step (c) further comprises incubating the extracted protein from step (b) in the buffer reservoir with Ubx protein in a ratio of 10 percent LOO_GFP-Ubx fusion protein to 90 percent Ubx protein.

18. The method of claim 17, wherein the LOO_GFP-Ubx is LOO8_GFP-Ubx and the left-out peptide is s8.

19. The method of claim 17, wherein the LOO_GFP-Ubx is LOO11_GFP-Ubx and the left-out peptide is s11.

20. The method of claim 17, wherein the denaturing wash includes a detergent and a concentration of 1M NaCl to 1.25M NaCl.


SEQUENCE LISTING (6 sequences)

SEQ ID NO: 1 - protein, 233 aa, Synthetic Construct
HEDGSVQLADHYQQNTPIGDGPVLLPDNHYLSTQTVLSKDPNEKRDHMVLLEFVTAAGITHGMDELYKGGTGGSMASKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQK

SEQ ID NO: 2 - protein, 629 aa, Synthetic Construct
MGHHHHHHHHHHSSGHEDGSVQLADHYQQNTPIGDGPVLLPDNHYLSTQTVLSKDPNEKRDHMVLLEFVTAAGITHGMDELYKGGTGGSMASKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKGSNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 3 - protein, 13 aa, Synthetic Construct
NGIKANFTVRHNV

SEQ ID NO: 4 - protein, 237 aa, Synthetic Construct
GITHGMDELYKGGTGGSMASKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVSSGHEDGSVQLADHYQQNTPIGDGPVLLPDNHYLSTQTVLSKDPNEKR

SEQ ID NO: 5 - protein, 631 aa, Synthetic Construct
MGHHHHHHHHHHSSGGITHGMDELYKGGTGGSMASKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVSSGHEDGSVQLADHYQQNTPIGDGPVLLPDNHYLSTQTVLSKDPNEKRNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 6 - protein, 12 aa, Synthetic Construct
DHMVLLEFVTAA